(19) United States
(12) Patent Application Publication
(10) Pub. No.: US 2012/0276602 A1
(43) Pub. Date: Nov. 1, 2012

(54) Method For Treatment of Oil

(75) Inventors: Kaare Joergensen, Dyssegaard (DK); Hanna Maria Lilbaek, Copenhagen Oe (DK); Lars Saaby Pedersen, Farum (DK); David Cowan, Chesham Buckinghamshire (GB); Hans Christian Holm, Hellerup (DK); Hon Seng Yee, Cheras (MY); Jan Hemann, Roedovre (DK)

(73) Assignee: Novozymes A/S, Bagsvaerd (DK)

(21) Appl. No.: 13/502,167

(22) PCT Filed: Oct. 19, 2010
(86) PCT No.: PCT/EP2010/065701

§ 371 Date: Jul. 10, 2012

(30) Foreign Application Priority Data
Oct. 21, 2009 (EP) 09173676.9

(51) Int. Cl. C12P 7/64

(52) U.S. Cl. 435/134; 428/402

(57) ABSTRACT

The present invention relates to a method for treatment of oil with a lipolytic enzyme which comprises contacting of the oil with particles of base-containing porous amorphous silica.

### FIELD OF THE INVENTION

The present invention relates to treatment of oil with a lipolytic enzyme.

### BACKGROUND OF THE INVENTION

Crude glyceride oils, particularly vegetable oils, are refined by a multi-stage process, the first step of which is usually degumming typically by treatment with water or with a chemical such as phosphoric acid or citric acid. After degumming, the oil may be refined by further chemical and/or physical processes including neutralization, bleaching and deodorizing steps.

EP 0507217A1 describes use of base-treated inorganic porous adsorbents for removal of contaminants such as phospholipids, metal ions and free fatty acids from glyceride oil. The process is disclosed for initial refining applications to replace or reduce the use of clay or bleaching earth, and, in particular, for reclamation applications of, e.g., spent frying oil. The adsorbents are characterized by being finely divided, i.e., they preferably are comprised of particles in the range from about 10 to about 100 micrometer. The base-treated adsorbents preferably are used wet to improve filterability.

Often vegetable or animal oils are used as blends in order to give the right physical and chemical properties in a given application. Furthermore the oils or blend of oils often need further processing to obtain suitable properties (e.g., melting profile, crystallization characteristics, mouth feel etc.). Such properties are often adjusted by rearranging or redistributing the fatty acids on the glycerol backbone either chemically or enzymatically. The exchange of one or more acyl groups among triglycerides is often referred to as “interesterification”. Enzymatic interesterification is carried out using a lipase.

In general, high enzyme productivity in such a process, e.g. enzymatic interesterification, can be obtained when operating with incoming oil of very high quality. But in practice, the enzyme activity within a reactor gradually decreases during use and the rate of activity decline has been shown to be closely linked to the quality of the incoming oil. In particular, presence of inorganic acids, small organic acids and oxidation compounds in the oil seems to negatively affect the working life of a lipase to be used (see, e.g., Holm and Cowan (2008), Eur. J. Lipid Sci. Technol. 110, pp. 679-691).

WO 2007/033013 describes a process for enzymatic interesterification of oil containing one or more metal chelating agents comprising the steps of: (a) contacting the oil with a base and (b) reacting said oil with a lipase.

JP2722600B2 describes treatment of oil with a lipase, where the half-life of the lipase is prolonged by adding sodium hydroxide treated celite to the oil.

WO 2008/069804 describes a continuous process for enzymatic treatment of oil where the oil is contacted with a processing aid before passing it through a plurality of enzyme-containing fixed bed reactors connected to one another in series. The processing aid can be substantially moisture-free silica which is preferably, when analyzed on a dry basis, at least 95% SiO₂, more preferably at least 99% SiO₂. To avoid the formation of soap in the reactor, it is preferred that the silica has a pH of less than about 7.0, and a pH of about 6.8 is particularly preferred.

Despite various attempts, there is still a need for industrially applicable methods for treatment of oil with a lipolytic enzyme, where the productivity of the enzyme is high even when the incoming oil may comprise impurities.

The object of the present invention is to provide a method for treatment of oil with a lipolytic enzyme wherein the productivity of the enzyme is less affected by impurities in the incoming oil. It is a further object to provide a method for efficiently neutralizing water-soluble acids in oil without excessive soap formation. It is a further object that such method shall be compatible with industrial processes for enzymatic treatment of oil used today.

### SUMMARY OF THE INVENTION

The present inventors have surprisingly found that when oil to be treated with a lipolytic enzyme is contacted with particles of base-containing porous amorphous silica, a dramatic increase in the working life of the enzyme is seen. When the base is contained in such particles, it is easy to handle and dose correctly, and the porosity of the particles ensures that the oil is brought sufficiently into contact with the base. Further, the inventors have found that the contacting of the oil with the particles of base-containing porous amorphous silica results in neutralization of water-soluble acids in the oil, whereas treatment with non-base-treated silica particles does not result in such neutralization. Further, the neutralization with the base-containing particles of the invention occurs without excessive soap formation. Soap formation leads to oil loss due to triglycerides being converted to soaps and because triglyceride oil is entrained in the soapstock that has to be removed. Thus, soap formation must be kept at a minimum both for enzyme reactions carried out in a batch process and for enzyme reactions carried out in a continuous mode of operation, e.g., as described in WO2008/069804. Furthermore, for enzyme reactions carried out as continuous operations in column reactors, a low level of soap is important to avoid column blockage, excessive pressure drop across the column and/or column replacement before all the enzyme activity is utilised.

The inventors have further found that base-containing porous amorphous silica particles having an average size of above 150 micrometer are particularly useful in such method. In a batch reaction, use of particles having a certain size may allow for rapid settling and also easier filtration. And also for continuous operations, e.g., in a packed bed reactor, particles which are too small are not very suitable, as the small particle size may result in a high pressure drop across the column. Further, the larger particle size may have a positive impact on the amount of soap produced, possibly because of the larger particles having a smaller surface area.

The present invention therefore relates to a method for treatment of oil containing water-soluble acids comprising the steps of:
a) contacting the oil with particles of base-containing porous amorphous silica having an average particle size of above 150 micrometer, and
b) reacting said oil with a lipolytic enzyme.

In a preferred embodiment of the method, said particles of base-containing porous amorphous silica have an average size of above 200 micrometer, preferably above 300 micrometer.

The invention further relates to particles of base-containing porous amorphous silica having the following properties:
a) an average particle size of above 150 micrometer,
b) an average pore diameter of 20-5,000 Angstroms,
c) a surface area of 10-1,200 m²/g, and
d) a moisture content of less than 30%,
   wherein the amount of base is 0.5-50 wt. %.

### DETAILED DESCRIPTION OF THE INVENTION

The present invention relates to a method for treatment of oil containing water-soluble acids comprising the steps of:
a) contacting the oil with particles of base-containing porous amorphous silica having an average particle size of above 150 micrometer, and
b) reacting said oil with a lipolytic enzyme.

### Oil

Any oil of vegetable or animal origin comprising fatty acids may be used in the method of the present invention. Fatty acids (FA) are in the context of the invention defined as free fatty acids (FFA) and/or fatty acid residues. Fatty acid residues may be present in polar lipids such as phospholipids; in non-polar or apolar lipids such as triglycerides, diglycerides, and monoglycerides; and/or in esters comprising fatty acids such as sterol esters or stanol esters.

The method described herein can be used for the treatment of any oil comprising fatty acids, whether edible or inedible. In some preferred embodiments, the oil is an edible oil.

In some embodiments the invention relates to a method, wherein the oil is a vegetable oil, for example canola oil, castor oil, cocoa butter, coconut oil, coriander oil, corn oil, cotton-seed oil, flax seed oil, jatropha oil, jojoba oil, hazelnut oil, hempseed oil, linseed oil, mustard oil, olive oil, palm oil, palm kernel oil, peanut oil, rapeseed oil, rice bran oil, safflower oil, sasanqua oil, shea butter, soybean oil, sunflower seed oil, tall oil or tsubaki oil. The oil may be or comprise any variety of “natural” oils having altered fatty acid composition, e.g. obtained via genetic modification or traditional “breeding”, such as high oleic or low linolenic, low saturated oils (e.g., high oleic canola oil, low linolenic soybean oil or high stearic sunflower seed oil).

In some embodiments the invention relates to a method, wherein the oil is of animal origin, for example butterfat, chicken fat, lanolin, lard, tallow, menhaden, fish liver oil or fish oil. The oil may be a by-product, such as from the production of omega-3 fatty acids from fish oil.

The treatment of algae oil is contemplated as well.

Also, blends and fractions of any of the above are included, such as palm olein or palm stearine, as well as above oils partially or fully hydrogenated. In one embodiment the oil is a blend of palm stearine and palm kernel oil, or a blend of palm stearine and coconut oil. In another embodiment the oil is a blend of fully hydrogenated soy bean oil (“Soy Flakes”) blended into soy bean oil.

The oil may be of any quality such as crude, refined, degummed, bleached and/or deodorized or any combination of these. For instance, refined oil may be prepared by treating with 0.05-0.1% phosphoric acid to remove gums at a temperature of 60-90° C. for 10-30 minutes. Bleached oil may be prepared by degumming with 0.05-0.1% phosphoric acid, followed by bleaching with 1% of bleaching earth at 105-110° C. for 15-30 minutes and filtration to remove the bleaching earth. Activated bleaching earth may be processed with sulfuric or hydrochloric acid.

In a preferred embodiment, the oil is vegetable oil which has been degummed. In another preferred embodiment, the oil is vegetable oil which has been refined. In another preferred embodiment, the oil is vegetable oil which has been bleached. In another preferred embodiment, the oil is vegetable oil which has been degummed and bleached. In another preferred embodiment, the oil is vegetable oil which has been refined and bleached. In another preferred embodiment, the oil is vegetable oil which has been refined, bleached and deodorized. In one embodiment, the oil is preferably not spent frying oil.

In a preferred embodiment, the oil has a content of free fatty acids which is below 0.3 wt. %. In a more preferred embodiment, the oil has a content of free fatty acids which is below 0.1 wt. %. In an even more preferred embodiment, the oil has a content of free fatty acids which is below 0.05 wt. %.

In some embodiments, the oil may comprise one or more short-chain alcohols. A short-chain alcohol is an alcohol having 1 to 5 carbon atoms (C1-C5) like, e.g., short-chain primary alcohols such as methanol, ethanol, propanol, butanol, and pentanol; and short-chain secondary alcohols such as isopropanol.

In some embodiments, a short-chain alcohol selected from the group consisting of methanol, ethanol, propanol, butanol, pentanol, isopropanol, or any combination thereof is added to the oil.

In one embodiment, the invention relates to a method where such short-chain alcohol is added to the oil after step a) but before step b). In another embodiment, the alcohol is added to the oil before step a).

Oil in the context of the present invention is to be interpreted broadly to encompass oils in the form of viscous liquids as well as oils which are merely in the form of liquefiable substances. Oil in the context of the present invention may be in a viscous liquid state (“oily”) at ambient temperatures or slightly warmer, but it may also be a liquefiable substance at ambient temperatures which becomes a viscous liquid when heated to a higher temperature, such as, e.g., 40° C., 50° C., 60° C., 70° C. or 80° C.

The oil to be treated according to a method of the present invention contains water-soluble acids. Water-soluble acids in the context of the present invention are weak or strong organic or inorganic acids which at least to some extent dissolve in water to form a homogeneous solution. Examples of water soluble acids include citric acid, phosphoric acid, sulphuric acid, hydrochloric acid, nitric acid and acetic acid. Water soluble acids may be present in the oil, e.g., as a residual from acid degumming or acid activated bleaching earth, or added as antioxidants. They dissolve in the small amounts of water normally present in the oil (100-500 ppm) and are taken up by the enzyme particles which are hydrophilic.

The presence of water-soluble acids may be determined by making a water extract of the oil and measuring the pH of this extract. If the oil contains water-soluble acids, i.e., inorganic acids and/or water-soluble organic acids such as citric acid or acetic acid, the pH of the water extract will be below 7.

In a preferred embodiment, the oil to be treated has a water extract pH below 6.5, preferably below 6, more preferably below 5.5 or below 5, and most preferably below 4.5. A ‘water extract pH’ in the context of the present invention means the pH of an extract of the oil made by (i) mixing vigorously the oil with a solution of 1% KCl in water in a w/w ratio of 3:1 (oil:KCl-solution), (ii) incubating for 1 hour at 70° C. to 75° C., and (iii) separating the two phases. pH is measured in the water phase.

In a preferred embodiment, the water-soluble acids contained in the oil are one or more of citric acid, phosphoric acid, sulphuric acid, hydrochloric acid, nitric acid and acetic acid.

### Particles of Base-Containing Porous Amorphous Silica

In the method according to the present invention, oil is contacted with particles of base-containing porous amorphous silica having an average particle size of above 150 micrometer.

The particles of base-containing porous amorphous silica preferably have an average size from 150 micrometer to 5,000 micrometer. In a preferred embodiment, the particles have an average size of above 200 micrometer, preferably above 250 micrometer, more preferably above 300 micrometer, and most preferably above 350 micrometer, above 400 micrometer, above 450 micrometer or above 500 micrometer. In another preferred embodiment, the particles have an average size of below 4,000 micrometer, preferably below 3,000 micrometer, more preferably below 2,000 micrometer, and most preferably below 1,000 micrometer. The particle size may be determined by sieving or by laser diffraction.

The particles of base-containing porous amorphous silica preferably have surface areas in the range from about 10 to about 1,200 m²/gram, more preferably from about 50 to about 400 m²/gram.

The particles of base-containing amorphous silica preferably have a porosity which makes them capable of soaking up to at least about 20 percent of their weight in moisture. In addition, the particles should contain at least some pores of sufficient size to permit access for the oil containing the water-soluble acids.

Untreated porous amorphous silica or other adsorptive materials can be blended with the particles of base-containing porous amorphous silica of the invention.

The term “amorphous silica” as used herein is intended to embrace silica gels, precipitated silicas, dialytic silicas and fumed silicas in their various prepared or activated forms.

In a preferred embodiment, the amorphous silica is precipitated silica, silica gel, dialytic silica or fumed silica. In a more preferred embodiment, the amorphous silica is precipitated silica or silica gel. In an even more preferred embodiment, the amorphous silica is precipitated silica.

The specific manufacturing process used to prepare the porous amorphous silica is not expected to affect its utility according to the invention.

Base treatment of the amorphous silica material selected for use according to the invention may be conducted as a step in the manufacturing process of the silica material or at a subsequent time. The base treatment process is described below.

Both silica gels and precipitated silicas are prepared by the destabilization of aqueous silica solutions by acid neutralization. In the preparation of precipitated silicas, the destabilization is carried out in the presence of inorganic salts, which lower the solubility of silica and cause precipitation of hydrated silica. The precipitate typically is filtered, washed and dried. In the preparation of silica gel, a silica hydrogel is formed which then typically is washed to low salt content. The washed hydrogel may be milled, or it may be dried ultimately to the point where its structure no longer changes as a result of shrinkage. The dried, stable silica is termed a “xerogel” if slow dried and termed an “aerogel” when quick dried. The aerogel typically has a higher pore volume than the xerogel. For preparation of precipitates, aerogels, or xerogels useful in this invention, it is preferred to dry them and then to add water to reach the desired water content for the particle sizing process which may be performed to obtain particles having the preferred average size from 150 micrometer to 5,000 micrometer. The larger particles may be dried and possibly screened to obtain the desired average particle size from 150 micrometer to 5,000 micrometer, more preferably of more than 200 micrometer. However, it is possible to initially dry the silica gel or precipitated silica to the desired water content, while forming the particles of the desired size in one step. These particles are then dried and possibly screened to obtain the desired average particle size and porosity.

Dialytic silica may be prepared by precipitation of silica from a soluble silicate solution containing electrolyte salts (e.g., NaNO₃, Na₂SO₄, KNO₃) while electro dialyzing as described in U.S. Pat. No. 4,508,607. Fumed silicas (or pyrogenic silicas) may be prepared from silica tetrachloride by high-temperature hydrolysis, or other convenient methods.

In some preferred embodiments of this invention, the particles of base-containing porous amorphous silica are prepared from precipitated silica. The inventors have found that precipitated silica particles effectively provides a large surface area, which allows for rapid removal of water-soluble acids from the oils when base is located in the porous structure of the particles. Further, the inventors have found that porous precipitated silica is particularly suitable for forming particles having a particle size which allows rapid settling in batch reactors and even allows fixed bed reactor operation without excessive pressure drop. The selection of precipitated silica is therefore particularly useful for facilitating the overall method for treatment of oil.

It is preferred that the base-containing particles to be used according to the present invention have the highest possible surface area in pores which are large enough to permit access to the oil containing water soluble acids; while still being capable of maintaining good structural integrity upon contact with the base and with the fluid media. Structural integrity is particularly important where the particles of base-containing porous amorphous silica are used in continuous or batch flow systems, where interaction between the silica particles and the processing equipment may cause degradation of the base-containing silica material. Amorphous silicas suitable for use in base-containing porous particles in such process preferably have surface areas of up to about 1,200 m²/g, more preferably between 10 and 1,200 m²/g, even more preferably from about 50 to about 400 m²/g. Preferably as much as possible of the surface area is contained in pores with diameters greater than 10-20 Angstroms. Preferably, the average pore diameter is 20-5,000 Angstroms, more preferably 100-2000 Angstroms. Particles with smaller pore diameters may be used, though.

One convention which describes porous amorphous silica materials is the average (volume) median pore diameter (“AVPD”), typically defined as that pore diameter at which 50 percent of the pore volume is contained in pores with diameters greater than the stated AVPD and 50 percent is contained in pores with diameters less than the stated AVPD. Thus, in porous amorphous silica materials suitable for use in the method of this invention, at least 50 percent of the pore volume is preferably in pores of at least 10-20 Angstroms, more preferably larger than 100 Angstroms, in diameter. Amorphous silica with a higher proportion of pores with diameters greater than about 100 Angstroms will be preferred, as these will allow for easy access of the acid-containing oil to the base located in the porous structure of the base-treated silica particles. The practical upper AVPD limit is about 5000 Angstroms.

Amorphous silica materials which have measured intraparticle AVPDs within the stated range will be suitable for use in the method of the invention. Alternatively, the required porosity may be achieved by the creation of an artificial pore network of interparticle voids in the 20 to 5000 Angstrom range. For example, non-porous silicas (i.e., fumed silica) can be used as aggregated particles. Silica materials, with or without the required porosity, may be used under conditions which create this artificial pore network. Thus, the criterion for selecting suitable porous silica materials for use in this process is the presence of an “effective average pore diameter” greater than 10-20 Angstroms, preferably greater than 100 Angstroms. This term includes both measured intraparticle AVPD and interparticle AVPD, designating the pores created by aggregation or packing of silica material particles.

The AVPD value (in Angstroms) can be measured by several methods. Both nitrogen and mercury porosimetry may be used to measure pore volume in for example precipitated silicas, xerogels, dried hydrogels, and dialytic silicas. Pore volume may be measured by the nitrogen Brunauer-Emmett-Teller (“B-E-T”) method described in Brunauer et al., J. Am. Chem. Soc., Vol. 60, p. 309 (1938). This method depends on the condensation of nitrogen into the pores of silica and is useful for measuring pores with diameters up to about 600 Angstroms. If the sample contains pores with diameters greater than about 600 Angstroms, the pore size distribution, at least of the larger pores, is determined by mercury porosimetry as described in Ritter et al., Ind. Eng. Chem. Anal. Ed. 17, p. 787 (1945). This method is based on determining the pressure required to force mercury into the pores of the sample. Mercury porosimetry, which is useful from about 30 to about 10,000 Angstroms, may be used alone for measuring pore volumes in silicas having pores with diameters both above and below 600 Angstroms. Alternatively, nitrogen porosimetry can be used in conjunction with mercury porosimetry for these silicas. For measurement of AVPDs below 600 Angstroms, it may be desired to compare the results obtained by both methods.

The surface area (SA) of silica materials to be used according to the invention can also be measured by the nitrogen B-E-T surface area method, described in the Brunauer et al., article, supra, or by the mercury porosimetry method. The surface area of all types of silica materials can be measured by these two methods; however, the BET method is the most common.

Moisture content (wt. %) of amorphous silica materials as well as base-treated amorphous silica may be determined by drying a sample in an oven at 105° C. for 12 to 24 hours and applying the following equation which uses the wet and dry weight differential:

Moisture content,wt. %=100*[(sample(as is,g)−sample(dry,g))/sample(as is,g)] (1)

The purity of the silica material to be used in the invention is not believed to be critical in terms of removing water-soluble acids from the oil. However, where the finished product is intended to be food grade oil, care should be taken to ensure that the base-treated porous amorphous silica used does not contain leachable impurities which could compromise the desired purity of the oil product. It is preferred, therefore, to use a substantially pure amorphous silica material. Minor amounts, i.e., less than about 10 percent, such as less than 8%, less than 5% or less than 3%, of other inorganic constituents may be present in the silica materials. For example, suitable silicas may comprise iron as Fe₂0₃, aluminum as Al₂0₃, titanium as TiO₂, calcium as CaO, sodium as Na₂O, zirconium as Zr0₂, and/or trace elements.

In a preferred embodiment, the base-treated porous amorphous silica particles to be used in the method of the invention are prepared from an amorphous silica material which is at least 55 wt. % SiO₂, preferably at least 65 wt. % SiO₂, more preferably at least 75 wt. % SiO₂, even more preferably at least 85 wt. % SiO₂, and most preferably at least 95 wt. % SiO₂. In another preferred aspect, the particles are prepared from an amorphous silica material which is about 98 wt. % SiO₂.

It is understood that the base-treated porous amorphous silica particles of this invention may be used alone or in combination with untreated porous silica materials or other types of porous supports (base-treated or not) useful for removing water-soluble acids or other impurities, which may be present.

The particles of the amorphous silica material have been treated with a base in such a manner that at least a portion of said base is retained in at least some of the pores of said silica material, thus resulting in the particles of base-containing porous amorphous silica to be used according to the method of the invention. Any base may be used in the formation of the base-containing particles. The base may be a strong base or a weak base or a combination of strong and weak bases. Care should be taken though when selecting the base and the base concentration, that it will not have any substantially adverse affect on the structural integrity of the silica material. Some structural change to the silica due to addition of the base is acceptable though in the method of the present invention.

In some embodiments of the invention, the base is a strong base. Preferably, the strong base is selected from the group consisting of sodium hydroxide (NaOH), potassium hydroxide (KOH), calcium hydroxide (Ca(OH)₂), magnesium hydroxide (Mg(OH)₂), and any combination thereof. More preferably, the strong base is selected from the group consisting of sodium hydroxide, potassium hydroxide, and a combination thereof.

In some embodiments of the invention, the base is a weak base. Preferably, the weak base is selected from the group consisting of sodium citrate, sodium lactate, sodium carbonate (Na₂CO₃), sodium hydrogen carbonate (NaHCO₃), disodium hydrogen phosphate, sodium dihydrogen phosphate, trisodium phosphate, sodium polyphosphate, potassium citrate, potassium lactate, potassium carbonate (K₂O₃), potassium hydrogen carbonate (KHCO₃), dipotassium hydrogen phosphate, potassium dihydrogen phosphate, tripotassium phosphate, potassium polyphosphate, calcium carbonate, ammonium carbonate, and any combination thereof. More preferably, the weak base is selected from the group consisting of sodium carbonate (Na₂CO₃), sodium hydrogen carbonate (NaHCO₃), potassium carbonate (K₂O₃), potassium hydrogen carbonate (KHCO₃), and any combination thereof.

It is desired that at least a portion of the pores in the particles contain base. When using solutions of strong base, possible interaction between the base and the silica material must be considered. For example, strong base in higher concentrations may cause changes to a silica support. Therefore, strong base should be used at lower concentration levels and dried quickly.

As stated, particles of amorphous silica material can be treated with a base in any manner that allows the base to enter at least a portion of the pores. For example, the silica particles, either finely divided or in their final particle size, may be suspended in the base or base solution for long enough time for the base or solution to enter at least a portion of the pores of the silica material, typically a period of at least about one half hour, up to about twenty hours. The slurry preferably will be agitated during this period to increase entry of the base into the pore structure of the silica material. The base-containing particles are then conveniently separated from the solution by filtration, and subsequently dried to the desired water content and then subjected to a particle sizing process, which may be performed to obtain particles having the preferred average size from 150 micrometer to 5,000 micrometer. These larger particles may then be further dried and possibly screened to obtain the desired average particle size, preferably from 150 micrometer to 5,000 micrometer, more preferably of more than 200 micrometer. Alternatively, the base solution can be introduced to the particles of the silica material in a fixed bed configuration for a similar period of contact. This may be conducted both on finely divided silica particles prior to a particle sizing process, or on silica particles, which already have the desired final particle size, preferably from 150 micrometer to 5,000 micrometer, more preferably of more than 200 micrometer.

A preferred method for producing base-treated particles of porous amorphous silica to be used according to the present invention is to impregnate the particles, which are either finely divided or in their final particle size, with a solution of base to about 70 percent to 100 percent (saturated) incipient wetness. Incipient wetness refers to the percent absorbent capacity of the silica material which is used. The finely divided base-treated silica particles may then undergo a particle sizing process, which may be performed to obtain base-treated porous amorphous silica particles having the preferred average size from 150 micrometer to 5,000 micrometer. These larger particles may then be dried and possibly screened to obtain the desired average particle size, preferably from 150 micrometer to 5,000 micrometer, more preferably of more than 200 micrometer. For example flash dried or spray dried precipitated silica particles may be treated in this manner either before or after the particle sizing process. Another method for the base-treatment is to introduce a fine spray or jet of the base solution to the silica material, preferably as it is fed to a particle sizing operation, or fed to the silica material after the particle sizing operation. For this method, it will be preferred to use a concentrated base. These methods may be preferred for treating amorphous silica in a commercial scale operation.

The particle sizing process mentioned above may be performed in any way as to create inert amorphous silica material as well as base-treated amorphous silica particles with the desired average particles size from 150 micrometer to 5,000 micrometer, more preferably of more than 200 micrometer. A non-exhaustive list of examples of such particle sizing processes include; granulation, agglomeration, roller compaction, extrusion, and milling.

The particles of base-containing porous amorphous silica are preferably dried to a moisture content of less than 30%, preferably less than 15%, preferably less than 10%, and more preferably less than 8%. Use of particles having such low moisture content may be advantageous as it may result in less formation of soap and also in a reduced loss of oil as a result of hydrolysis as compared to a similar process using particles having higher moisture content.

The content of base in the particles of the base-containing porous amorphous silica to be used according to the present invention is preferably 0.5-50 wt. %. More preferably, the content of base in the particles is 1-30 wt. %, even more preferably 5-30 wt. %, and most preferably 10-25 wt. %.

The particles of base-containing porous amorphous silica to be used according to the invention preferably have a pH of more than 7.5, e.g., a pH of more than 7.8, more preferably a pH of more than 8, e.g., a pH of more than 8.5 or more than 9. The pH of the particles may be determined by making, e.g., a 5% suspension of the particles in water and measuring the pH of the aqueous suspension.

### Lipolytic Enzyme

In step b) in above method according to the present invention, oil is reacted with a lipolytic enzyme as catalyst.

The lipolytic enzyme may be immobilized.

In a preferred embodiment, the lipolytic enzyme is not immobilized on the particles of base-containing porous amorphous silica referred to in step a). In that case, if immobilized, the lipolytic enzyme may optionally be immobilized on the same type of porous amorphous silica material as that which contains the base, but then the enzyme is immobilized onto separate silica material, such as separate particles of silica material. This is an advantage since the stability of the enzyme may be affected if it is immobilized onto the base-containing particles. Also, it allows for varying the ratio of base to enzyme as needed, e.g., depending on which oil is to be treated, and it allows for loading the base-containing silica particles and the enzyme into separate reactors or into the same reactor where the base-containing particles are loaded on top of the enzyme.

A lipolytic enzyme in the context of the present invention may be an enzyme which is capable of hydrolyzing carboxylic ester bonds to release carboxylate (EC 3.1.1). The lipolytic enzyme is an enzyme classified under the Enzyme Classification number E.C. 3.1.1.—(Carboxylic Ester Hydrolases) in accordance with the Recommendations (1992) of the International Union of Biochemistry and Molecular Biology (IUBMB). Thus, the lipolytic enzyme may exhibit hydrolytic activity, typically at a water/lipid interface, towards carboxylic ester bonds in substrates such as mono-, di- and triglycerides, phospholipids, thioesters, cholesterol esters, wax-esters, cutin, suberin, synthetic esters or other lipids mentioned in the context of E.C. 3.1.1. The lipolytic enzyme may, e.g., have triacylglycerol lipase activity (EC 3.1.1.3, 1,3-positionally specific or non-specific), phospholipase activity (A1 or A2, EC 3.1.1.32 or EC 3.1.1.4), esterase activity (EC 3.1.1.1) or cutinase activity (EC 3.1.1.74).

Suitable lipolytic enzymes (e.g., lipases) include those of bacterial or fungal origin. Chemically modified or protein engineered mutants are included. Examples include lipases from Candida, such as C. antarctica (e.g., lipases A and B described in WO 88/02775), C. rugosa (C. cylindracea); Rhizomucor, such as R. miehei; Hyphozyma; Humicola; Thermomyces, such as T. lanuginosus (H. lanuginosa lipase) as described in EP 258 068 and EP 305 216; a Pseudomonas lipase, e.g., from P. alcaligenes or P. pseudoalcaligenes (EP 218 272), P. cepacia (EP 331 376), P. glumae, P. stutzeri (GB 1,372,034), P. fluorescens, Pseudomonas sp. strain SD 705 (WO 95/06720 and WO 96/27002), P. wisconsinensis (WO 96/12012); a Bacillus lipase, e.g., from B. subtilis (Dartois et al. (1993), Biochemica et Biophysica Acta, 1131, 253-360), B. stearothermophilus (JP 64/744992) or B. pumilus (WO 91/16422); lipase/phospholipase from Fusarium oxysporum; lipase from F. heterosporum; lysophospholipase from Aspergillus foetidus; phospholipase A1 from A. oryzae; lipase from A. oryzae; lipase/ferulic acid esterase from A. niger; lipase/ferulic acid esterase from A. tubingensis; lipase from A. tubingensis; lysophospholipase from A. niger; and lipase from F. solani.

The lipolytic enzyme may be positionally site specific (e.g., 1,3 specific) or non-specific, upon interaction with triglycerides as substrates.

Furthermore, a number of cloned lipolytic enzymes may be useful, including the Penicillium camembertii lipase described by Yamaguchi et al., (1991), (Gene 103, 61-67), the Geotricum candidum lipase (Shimada, Y. et al., (1989), J. Biochem., 106, 383-388), and various Rhizopus lipases such as a R. delemar lipase (Hass, M. J et al., (1991), Gene 109, 117-113), a R. niveus lipase (Kugimiya et al., (1992), Biosci. Biotech. Biochem. 56, 716-719) and a R. oryzae lipase.

Other types of lipolytic enzymes such as cutinases may also be contemplated, e.g., cutinase from Pseudomonas mendocina (WO 88/09367), Fusarium solani pisi (WO 90/09446) or H. insolens (U.S. Pat. No. 5,827,719).

The enzyme may be an enzyme variant produced, for example, by recombinant techniques. Examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079 and WO 97/07202.

Examples of commercially available lipases include Lipex™, Lipoprime™, Lipolase™ Lipolase™ Ultra, Lipozyme™, Palatase™, Novozym™ 435 and Lecitase™ (all available from Novozymes A/S). Other commercially available lipases include Lumafast™ (Pseudomonas mendocina lipase from Genencor International Inc.); Lipomax™ (Ps. Pseudoalcaligenes lipase from DSM/Genencor Int. Inc.); and Bacillus sp. lipase from Genencor. Further lipases are available from other suppliers.

In a preferred embodiment, the lipolytic enzyme is a lipase (EC 3.1.1.3).

Preferably, the reaction with the lipolytic enzyme results in interesterification or transesterification of lipids in the oil. More preferably, the reaction with the lipolytic enzyme results in interesterification.

Interesterification in the context of the present invention may be a reaction which involves an exchange of one or more acyl groups among triglycerides. Acyl groups may exchange positions within a single triglyceride molecule or among different triglyceride molecules.

Transesterification in the context of the present invention may be the process of exchanging one or more acyl groups among different molecules, such as among different triglyceride molecules, or the process of transferring of an acyl group from, e.g., a triglyceride or a free fatty acid to an alcohol, e.g., to form a fatty acid alkyl ester. A common example of a transesterification is the reaction between a triglyceride and methanol to form fatty acid methyl ester (biodiesel) and diglyceride. The diglyceride can further react in other transesterifications.

Biodiesel represents a promising alternative fuel for use in compression-ignition (diesel) engines. Such biodiesel may in principle be any fatty acid alkyl ester of a short-chain alcohol, where a short-chain alcohol is an alcohol having 1 to 5 carbon atoms (C1-C5), and it may be produced by enzymatic transfer to the alcohol of fatty acid residues derived from triglycerides, diglycerides, or monoglycerides, or from esters comprising fatty acids such as sterol ester, stanol ester, or any combination thereof.

In some preferred embodiments, the lipolytic enzyme is immobilized. The use of immobilized enzymes in processing of oils experience significant growth due to new technology developments that have enabled cost effective methods. A fundamental advantage of immobilized enzymes is that they can be recovered and re-used from a batch process by simple filtration. Further, packing of immobilized enzymes in columns allows for easy implementation of a continuous process. Immobilized enzymes generally also have a positive effect on operational stability of the catalyst (compared to free enzymes), it makes handling easier (compared to free enzyme powder), and it allows operation under low-water conditions (compared to liquid formulated enzymes).

Various ways of immobilizing lipolytic enzymes are well known in the art. A review of lipase immobilization is found in “Immobilized lipase reactors for modification of fats and oils—a review” Malcata, F X., et al. (1990) J. Am. Oil Chem. Soc. Vol. 67 p. 890-910, where examples of representative lipase immobilizing carriers are illustrated, including inorganic carriers such as diatomaceous earth, silica, porous glass, etc.; various synthetic resins and synthetic resin ion exchangers; and natural polysaccharide carriers such as cellulose and cross-linked dextrin introduced with ion exchange groups.

In some embodiments the invention relates to a method, wherein the lipolytic enzyme is immobilized either on a carrier; by entrapment in natural or synthetic matrices, such as sol-gels, alginate, and carrageenan; by cross-linking methods such as in cross-linked enzyme crystals (CLEC) and cross-linked enzyme aggregates (CLEA); or by precipitation on salt crystals such as protein-coated micro-crystals (PCMC).

In some embodiments the invention relates to a method, wherein the carrier is a hydrophilic carrier selected from the group containing: porous inorganic particles composed of alumina, silica or silicates such as porous glas, zeolites, diatomaceous earth, bentonite, vermiculite, hydrotalcite; and porous organic particles composed of carbohydrate polymers such as agarose or cellulose.

In some embodiments the invention relates to a method, wherein the carrier is a hydrophobic carrier selected from the group containing: synthetic polymers such as nylon, polyethyllene, polypropylene, polymethacrylate, or polystyrene; and activated carbon.

Lipolytic enzymes in solid form, such as immobilized lipolytic enzymes, may be used in some embodiments of the invention and examples of commercially available immobilized lipolytic enzyme include the ones sold under the trade names LIPOZYME TL IM™, LIPOZYME RM IM™, and Novozym 435 (Novozymes A/S).

In case the reaction is carried out with liquid formulations of lipolytic enzyme (in contrast to immobilized lipolytic enzyme), the enzyme can be recovered for multiple uses by either separation off the water/glycerol phase containing the enzyme or by using a membrane reactor. In a membrane reactor the end-product is separated from the lipase by using a membrane filtration system.

### Treatment of Oil

In above method according to the present invention, treatment of oil, e.g., an oil composition comprising one or more water-soluble acids, is performed comprising contacting the oil with particles of base-containing porous amorphous silica and reacting the oil with a lipolytic enzyme. Such oil treatment can be carried out in laboratory scale as well as industrial scale in a number of different ways.

The oil treatment according to the invention can be carried out in one reactor or in a series of two or more reactors. The treatment can be carried out in one or more batch reactors in series, in one or more continuous reactors in series or in a set up using a combination of batch and continuously operating reactors. The scope of the present invention is in no way limited by the type and the set up of the reactors and/or by the specific sequence of reactors that are used for the base treatment and the reaction catalyzed by the lipolytic enzyme.

Lipolytic enzymes can be applied dissolved in an aqueous solution, as a dried protein product or in an immobilized form. The scope of the invention is not limited by the form in which the lipolytic enzyme is applied or the type of immobilization method used.

The oil is contacted with particles of base-containing porous amorphous silica in order to reduce the negative effect of water-soluble acids on the productivity of the lipolytic enzyme. The extent to which such base treatment is needed is highly dependent on the type and quality of the oil, more specifically on the type and content of the water-soluble acid or acids. The skilled person will know how to determine the dosage of base-containing particles which is needed in a specific set-up. The dosage can be easily determined based on application testing, e.g., in laboratory scale, or it can be determined based on experience from prior use of such product on similar types of substrates. In general, a dosage of 1-5 kg of base-containing porous amorphous silica particles will be sufficient, e.g., for treatment of 8,000 kg oil, depending on the quality of the oil and the base content of the particles.

The contacting of the oil with base-containing particles according to the present invention in order to reduce the negative effect of water-soluble acids on the lipolytic enzyme can be made in different ways. The base-containing particles and the lipolytic enzyme can be used either in separate reactors or in a combined reactor which comprises both the particles of base-containing porous amorphous silica and the lipolytic enzyme. Also, contacting of the oil with the base-containing particles in intermediate stages between one or more reaction stages applying lipolytic enzyme is a viable configuration. The scope of the invention is not limited to any particular way of contacting the oil with the particles of base-containing porous amorphous silica.

The particles of base-containing porous amorphous silica may be contacted with the oil, e.g., in a batch type of treatment, in a continuously stirred tank reactor, in a series of continuously stirred tank reactors, in a fixed bed reactor, or in a series of fixed bed reactors.

When operating the reactors for treatment of the oil with the base-containing particles, it is important that the oil and the base-containing particles are given sufficient contact time to obtain the desired effect. However, the specific flow rate or the variations of the flow rate as well as variation in residence time is not important as long as the sufficient contact time is achieved. A sufficient contact time is achieved when the content of water-soluble acids in the oil has been sufficiently decreased. This may be determined by measuring pH in a water extract of the oil as described above. The treatment of the oil with the particles of base-containing porous amorphous silica cannot be overdone. Hence there is no limit on maximum reaction time.

In some embodiments of the method of the invention, the contacting of the oil with the particles of base-containing porous amorphous silica provides a pH in a water extract of the oil which is above pH 5, such as above pH 5.5 or above pH 6, such as about pH 7. Alternatively, the contacting of the oil with the particles of base-containing porous amorphous silica provides a pH in a water extract of the oil which is within the range of +/−1 around the pH optimum of the lipolytic enzyme used in the method of the invention.

In some embodiments of the method of the invention, the contacting of the oil with the particles of base-containing porous amorphous silica provides an increase in pH in a water extract of the oil of at least 0.2, preferably at least 0.5.

The contacting of the oil with the particles of base-containing porous amorphous silica and the reaction of the oil with the lipolytic enzyme may be carried out sequentially or simultaneously.

In some embodiments, these two steps are carried out simultaneously. They may be carried out as a process where the oil is passed through a treatment system comprising at least one reactor containing lipolytic enzyme, and wherein the particles of base-containing porous amorphous silica are mixed with the lipolytic enzyme in at least one such reactor. Such process may be a batch process or it may be a continuous process.

In some embodiments, the contacting of the oil with the particles of base-containing porous amorphous silica and the reaction of the oil with the lipolytic enzyme are carried out sequentially. They may be carried out as a process where the oil is passed through a treatment system comprising at least one reactor containing lipolytic enzyme, and wherein the particles of base-containing porous amorphous silica are loaded in at least one separate reactor. Such process may be a batch process or it may be a continuous process. In this set-up, the oil is passed through at least one reactor loaded with base-containing particles before it is passed through at least one reactor containing lipolytic enzyme. In a preferred embodiment, at least one separate reactor loaded with particles of base-containing porous amorphous silica is connected in series to the at least one reactor containing lipolytic enzyme.

Alternatively, the base treatment and the enzyme treatment may be carried out as a process where the oil is passed through a treatment system comprising at least one reactor containing lipolytic enzyme, and wherein the particles of base-containing porous amorphous silica are loaded on top of the lipolytic enzyme in at least one such reactor. Such process may be a batch process or it may be a continuous process.

In some preferred embodiments, the contacting of the oil with particles of base-containing porous amorphous silica and the treatment of the oil with a lipolytic enzyme are carried out in continuous mode of operation.

The present invention is further described by the following examples that should not be construed as limiting the scope of the invention.

### EXAMPLES

### Materials:

### Oils:

Blend of palm stearine and coconut oil (blend ratio 70:30 (w:w)), both RBD quality from Aarhus Karlshamn, Sweden (Examples 2, 5 and 6)
Blend of palm stearine and palm kernel oil (blend ratio 70:30 (w:w)), both RBD quality from Unimills, the Netherlands (Examples 3, 4, and 7)

### (RBD: Refined, Bleached and Deodorized)

### Enzyme:

Immobilized lipase A: Immobilized lipase derived from Humicola lanuginosa/Thermomyces lanuginose disclosed and produced recombinantly in Aspergillus oryzae as disclosed in EP 305216B1. The immobilization method is described in WO 09/010,561.

### Chemicals:

Chemicals used as buffers and substrates were commercial products of at least reagent grade. All chemicals, unless otherwise indicated, were obtained from Sigma-Aldrich or a similar commercial source and used without further purification.

Bases used in Example 1:
Potassium carbonate anhydrous (K₂O₃), min. 99% from J. T. Baker
Potassium bicarbonate (KHCO₃), min. 99.5% from Sigma-Aldrich
Sodium carbonate anhydrous (Na₂CO₃), min. 99.5% from Fluka Analytical
Potassium hydroxide (KOH) flakes/pellets, 85% KOH, 10-15% water, from Sigma-Aldrich

### Methods:

### Determination of Solid Fat Content (SFC)

The method used for determination of solid fat content is based on the AOCS Official Method Cd 16b-93 “Solid Fat Content (SFC) by Low-Resolution Nuclear Magnetic Resonance”. The solid fat content is given as %.

### Apparatus:

Oven—maintained at 80° C.
Cooling bath set at 0° C.
Constant temperature water baths (40° C. and 60° C.+/−0.1° C.)
Metal blocks (aluminum) with holes for SFC tubes
NMR tubes
NMR spectrometer, Minispec mq-series 2001, Bruker Optics Inc, TX, USA.

### Stopwatch

### SFC Procedure:

1 The fat blend is melted at 80° C. for 30 min (or microwave)
2 Fat blend (˜3 ml) is transferred to NMR tubes (duplicate tubes)
3 Place the tubes in 80° C. ˜5 min (if apparent solids in a tube)
4 The NMR tubes are transferred to water bath at 60° C. for 5 to 15 min.
5 The NMR tubes are transferred to cooling bath at 0° C. for 60+/−1 min
6 The NMR tubes are subsequently placed in water bath for 30 min at the chosen temperature, typically 40° C.
7 The NMR tubes are transferred to the cavity of the NMR spectrometer one by one and are measured as quickly as possible. The magnet in the NMR spectrometer is thermostated at 40° C.

Mineral oil samples for calibration of the NMR instrument is supplied by Bruker Optics Inc, TX, USA.

### Multiple Batch Assay (MBA Assay)

The method is used to determine performance of immobilized lipases for interesterification in Multiple Batch Reactions.

### Principle:

An oil blend is interesterified in a batch reaction using an immobilized lipase as catalyst. At the end of each batch reaction the oil is decanted from the catalyst which remains in the reactor. Then fresh oil is added to the catalyst and another batch reaction is carried out. The average reaction rate of the enzyme is determined from each batch reaction.

Based on the average reaction rate of the enzyme in a number of consecutive batch reactions with reuse of the enzyme, it is possible to estimate the enzyme deactivation rate as a function of oil volume that has been in contact with the enzyme.

Solid fat content (SFC) is used to quantify the change to the fat properties due to the interesterification.

### The Results:

Typically the experiments are used to:
Make direct side-by-side comparison of the performance of two or more immobilized enzyme products by looking at plots of either the solid fat content or the average reaction rate constant versus batch number or produced amount of oil per mass of immobilized enzyme.
To estimate the average production rate to a given productivity at constant conversion according to the model described below. The unit of the result is mass of oil interesterified per mass of immobilized enzyme per time.

### Apparatus:

Batch reactor: Duran Square bottles with pouring ring and screw cap. Capacity 250 ml.
Oven with orbital shaker: An oven that can keep the temperature constant at 70° C.+/−2° C. and which can be equipped with an orbital shaker. Shaking diameter: 25 mm. Shaking speed: 300 rpm.

### MBA Procedure:

1. Melt the oil and heat it to 70° C.
2. Tara weight an empty reaction flask
3. Weigh out 0.50 g immobilized enzyme sample in the reaction flask
4. Weigh out approx. 100 g oil in the reaction flask and note the exact weight
5. Flush the flask thoroughly with nitrogen and close the flask tightly
6. Incubate the flask in a shaker incubator at 70° C. for 24 h. Note the exact start time
7. After 24 h, take the reaction flask out of the incubator and place the flask on a weight. Take out sample for SFC measurement and decant the remaining oil in a waste container, taking care of not removing enzyme. Note the exact weight of oil removed
8. Repeat steps 4.-7 until nine batches of oil have been contacted with the enzyme

### Equilibrium for the Reaction:

In order to use the reaction model it is necessary to determine the solid fat content of the fat at the reaction equilibrium. This is done by making a batch reaction with high enzyme to oil ratio. In practice, the MBA procedure is carried out with 2.0 g enzyme and 100 g oil for 4 batches. The average SFC of the four batches is used as the equilibrium SFC value.

### Calculation of Results:

The kinetics for the reaction of Lipozyme TL IM is modeled using a first order reversible reaction model with solid fat content as the concentration parameter.

$$k = \frac{1}{τ} \cdot ln [ \frac{{SFC}_{i n} - {SFC}_{eq}}{{SFC}_{out} - {SFC}_{eq}} ] \qquad ( 1 )$$

### Where

k is the rate constant
τ is the weight based reaction time
SFCᵢₙ is the solid fat content of the oil that enters the reactor
SFCₒᵤₜ is the solid fat content of the oil that leaves the reactor.
SFC_{eq} is the solid fat content of the oil at the reaction equilibrium.
   For a fixed bed reactor

$$τ = \frac{w}{F} \qquad ( 2 )$$

and for a batch reactor

$$τ = \frac{w \cdot {t}_{b}}{{M}_{b}} \qquad ( 3 )$$

### Where

w is the mass of the catalyst—Lipozyme LT IM
F is the mass flow rate of oil through the reactor
M_{b} is the mass of oil in the reactor
t_{b} is the reaction time in the batch reactor
   An exponential model is used, to describe the rate constant as a function of the productivity

$${k}_{model} = {k}_{0} \cdot exp ( \frac{- ln ( 2 )}{{V}_{1 / 2}} \cdot V ) \qquad ( 4 )$$

### Where

k_{model} is the model of the rate constant
k₀ is the rate constant for the fresh enzyme
V_{1/2} is the volume based half life of the enzyme—the amount of oil per amount of enzyme that is needed to reduce k_{model} by 50%.
V is the amount of oil per amount of enzyme that has passed the reactor

The oil production rate and the productivity at any given point in time are related as stated in equation (5).

$$( \frac{F}{w} ) = \frac{d V}{d t} where V = 0 for t = 0 \qquad ( 5 )$$

### Where

t is the operation time of the reactor.

By combining the equations (1), (2), (4) and (5) and solve the resulting differential equation the following relations are obtained for the productivity and production rate at a given point in time for a fixed be reactor operating at constant conversion:

$$V = \frac{{V}_{1 / 2}}{ln ( 2 )} \cdot ln ( 1 - \frac{{k}_{0} - ln ( 2 )}{{V}_{1 / 2} \cdot ln ( \frac{{SFC}_{out} - {SFC}_{eq}}{{SFC}_{i n} - {SFC}_{eq}} )} \cdot t ) \qquad ( 6 )$$

and

$$( \frac{F}{w} ) = \frac{{k}_{0} \cdot {V}_{1 / 2}}{{k}_{0} \cdot ln ( 2 ) \cdot t - {V}_{1 / 2} \cdot ln ( \frac{{SFC}_{out} - {SFC}_{eq}}{{SFC}_{i n} - {SFC}_{eq}} )} \qquad ( 7 )$$

and the average production rate to a given productivity at constant level of conversion can be calculated according to (6):

$${( \frac{F}{w} )}_{avg} = \frac{{k}_{0} \cdot ln ( 2 )}{ln ( \frac{{SFC}_{out} - {SFC}_{eq}}{{SFC}_{i n} - {SFC}_{eq}} )} \cdot ( \frac{( \frac{{V}_{tot}}{{V}_{1 / 2}} )}{{2}^{( \frac{{V}_{tot}}{{V}_{1 / 2}} )} - 1} ) \qquad ( 8 )$$

### Where

$${( \frac{F}{w} )}_{avg}$$

is the average production rate
Vₜₒₜ is the productivity to which the average production rate is calculated.

Combining equation (1), (2), (4) and (8) and specifying the productivity, Vₜₒₜ, and SFCᵢₙ, a model is obtained for SFCₒᵤₜ which contain two parameters, V_{1/2} and

$${( \frac{F}{w} )}_{avg} .$$

These parameters can be determined by fitting the model to the experimental data for SFCₒᵤₜ versus V using non linear parameter estimation by minimizing the sum of squared differences between the SFCₒᵤₜ determined experimentally and the SFCₒᵤₜ calculated according to the model.

The confidence intervals are calculated using the profile likelihood method.

### Determination of Soap Content of Oil

The method is based on the AOCS Recommended Practice Cc 17-95.

### Apparatus:

### Microburette, 5 ml

Hotplate with magnetic stirring

### Reagents:

Acetone containing 2% water, prepared by adding 20 ml deionized water to 980 ml of reagent grade acetone
Hydrochloric acid (HCl), approximately 0.01 N, accurately standardized
Bromophenol blue indicator solution, 1.0% in water
Sodium hydroxide (NaOH), approximately 0.01N

### Procedure:

1 Just prior to the analysis, prepare the test solution by adding 0.5 ml of the bromophenol blue indicator solution to each 25 ml of the aqueous acetone solution and titrating with 0.01N HCl or 0.01N NaOH until the solution is just yellow in color.
2 Weigh 10 g of the oil into a 50 ml glass beaker which has been well rinsed with the test solution.
3 Add 250 microliter of water in the test sample, warm on a hotplate with vigorous magnetic stirring. Add 12.5 ml of the test solution and after thorough mixing allow the contents to separate into two distinct layers.
4 Slowly add 0.01N HCl from the microburet until the color just changes from green/blue to yellow. Repeat the warming, stirring and HCl addition until the yellow color of the upper layer remains permanent.
5 Record the total volume of the HCl used.
6 A blank correction is determined on soap-free oil using this same procedure, steps 1-5.

### Calculations:

The amount of soap is calculated as ppm sodium oleate or potassium oleate, depending on the base used in the experiment in question.

$$ppm soap as sodium oleate = \frac{( V ( sample ) - V ( blank ) ) \cdot N \cdot 304 \cdot 1000}{m ( sample )}$$

### Where

V(sample)=volume of HCl obtained for the sample in procedure step 5
V(blank)=volume of HCl obtained for the blank in procedure step 6
N=the molarity of the HCl solution
304.4=the molar weight of sodium oleate. When results are expressed as potassium oleate, the molar weight of potassium oleate, 342.5, is used instead.
m(sample)=weight of the oil sample, g

### Determination of FFA Content of Oil

The method is based on the AOCS Official method Ca 5a-40.

### Apparatus:

### Microburette, 5 ml

Hotplate with magnetic stirring

### Chemicals:

Sodium hydroxide, 1 N titrisol ampulle dissolved in 1 l deionized water and 2-propanol, 99.5% pure
Ethanol, 96% pure
Phenolphthalein, 99% pure, 1% solution prepared in 96% ethanol.

### Procedure:

1 Weigh out 1.0 g sample in a conical flask and note the exact weight. (Heat the sample gently if it solidifies)
2 Add 20 ml of 2-propanol
3 Shake the flask until the sample is completely dissolved. (Heat the sample gently if it solidifies)
4 Add 4-8 drops of phenolphthalein indicator and shake the flask
5 Make a blind sample consisting of 20 ml 2-propanol and 4-8 drops of phenolphthalein.
6 Titrate the sample(s) and the blank with 0.01 M NaOH until the first pale permanent pink color appears (color must persist for 30 sec.)
7 Note the NaOH amount for later calculation

### Calculations:

The calculation is based on oil systems where the predominant fatty acid is oleic acid (C18:1) and includes subtraction of a ‘blind sample’.

$$Free fatty acids as oleic , \% = \frac{( V ( sample ) - V ( blank ) ) \cdot N \cdot 28.2}{m ( sample )}$$

### Where

V(sample)=volume of NaOH obtained for the sample in procedure step 7
V(blank)=volume of HCl obtained for the blank in procedure step 7
N=the molarity of the NaOH solution
m(sample)=weight of the oil sample, g

### Determination of pH in Water Extract of Oil

### Apparatus:

Heating cabinet maintained at 75° C.
Balance with 0.01 g precision
Ultra-Turrax high shear mixer
pH electrode

### Procedure:

1 Heat up the oil until it becomes liquefied.
2 Adjust the pH of deionized water (Millipore) to (7.0±0.2) with 0.01N NaOH
3 Weigh 150 g of oil and 150 g of water into a 500 ml bottle.
4 Warm the mixture in the oven at 75° C. for one hour.
5 Homogenize the mixture with a high shear mixer, Ultra Turrax (24000 rpm) for 1½% minutes
6 Keep the homogenized mixture in the oven at 75° C. until the water layer separates at the bottom of the flask. Pipette out the water components.
7 Cool down the water to ambient temperature and measure the pH value.

A modified procedure was used in Examples 3, 4 and 7, where 15 g oil and 15 g water were mixed in step 3 and the homogenization time in step 5 was reduced to 30 seconds.

### Determination of Peroxide Value (PV)

The method is based upon AOCS Cd 8-53 Reapproved 1997 Peroxide Value (Acetic Acid-Chloroform Method)

### Apparatus

### Autotitrator Mettler Toledo DL 50 (Electrode DM 140-SC)

Titration beaker
Analytical balance
Disposable pipettes

### Dispenser

### Pipette 0.5 ml

### Reagent Bottle

### Procedure

A test portion is treated in a solution of acetic acid and chloroform with potassium iodide solution. The free iodine is titrated with standardized sodium thiosulphate solution. The peroxide value is a measure expressed in term of milliequivalents of active oxygen per 1 kg of sample that oxidize potassium iodide under the conditions of the test.

### Example 1

### Manufacture of Base-Containing Porous Amorphous Silica (Base on Silica)

The porous precipitated silica particles used to make the base-containing particles of this example were of average particle size 550-850 micrometer, moisture content of 4.5-6.2%, capable of soaking up about 140% of its weight in moisture, has an average median pore diameter (volume) of approx. 280 Angstroms, and a surface area (SA by BET) of about 165 m²/g.

The base-containing porous amorphous silica product was produced in the following manner:
1. A liquid solution of base and water was produced as per the data in Table 1, e.g. for product id. A: 186 g of KOH pellets (85% KOH) were dissolved in 3872 g of water.
2. The liquid solution of base and water (according to 1) was then applied onto 1050-3000 g of precipitated silica particles (exact amounts as per Table 1 below) in a 10 or 20 L mixer (Lödige, Germany) using continuous mixing with a rotating speed of 150 rpm at ambient temperature. An atomizing nozzle was used to distribute the liquid over the silica particles. Total spraying time was 10-20 minutes.
3. After addition of the liquid solution (According to 2), the moist carrier particles were dried in a fluidized bed (GEA MP-3/2/3) with inlet air temperature of approx. 90° C. until the product temperature reached 60-70° C. This drying time was 10-40 min and resulted in the moisture contents of the base-containing porous amorphous silica indicated by Table 1.

From Table 1 it is noted that base-containing porous amorphous silica of average particle size 575-808 micrometer is produced in this way. Thus, it is seen that substantially no particle size enlargement nor particle size degradation has occurred during the process of impregnating the precipitated silica particles with base.

TABLE 1
Base-containing porous amorphous silica particles were produced with the
following characteristics

| | | Amt. of base | D_{v,50}; Avg. | Moisture | | | Silica |
|---|---|---|---|---|---|---|---|
| | Base | in product, | part. size, | content, | Base | Water, | particles, |
| Id. | used | wt. % | μm | wt. % | added, g | g | g |
| A | KOH | 5% | 700 | 2.1 | 186 (85%) | 3872 | 3000 |
| B | KOH | 10% | 579 | 2.2 | 392 (85%) | 3000 | 3000 |
| C | Na₂CO₃ | 5% | 721 | 2.0 | 159 (99%) | 3898 | 3000 |
| D | Na₂CO₃ | 10% | 730 | 2.6 | 337 (99%) | 3897 | 3000 |
| E | KOH | 20% | 585 | 6.0 | 353 (85%) | 1200 | 1200 |
| F | Na₂CO₃ | 20% | 612 | 6.2 | 303 (99%) | 1200 | 1200 |
| G | KOH | 30% | 808 | 9.9 | 529 (85%) | 450 | 1050 |
| H | KHCO₃ | 20% | 587 | 3.1 | 303 (99%) | 1200 | 1200 |
| I | K₂CO₃ | 20% | 627 | 2.7 | 303 (99%) | 1200 | 1200 |
| J | K₂CO₃ | 5% | 575 | 2.4 | 75 (99%) | 1810 | 1425 |
| K | K₂CO₃ | 10% | 583 | 1.7 | 150 (99%) | 1675 | 1350 |
| L | None | n/a | 595 | 4.5 | n/a | n/a | n/a |
| M | None | n/a | 844 | 6.2 | n/a | n/a | n/a |

Average particle size, D_{v,50}, was determined by laser diffraction using a Malvern Mastersizer 2000 with a Scirocco 2000 dry feeder. An air dispersion pressure of 0.25 bar and a particle refractive index of 1.45 were used.

The base-treated porous amorphous silica particles produced in this example were applied in the following examples.

### Example 2

### Neutralization of Water Soluble Acids in Vegetable Oil Blend Using Base-Treated Silica

The purpose of this example was to study the neutralization of water soluble acids in a vegetable oil blend by batchwise treatment of oil with base-treated silica.

The oil blend consisting of 30% coconut oil and 70% palm stearine was heated to 70° C. and 1-25% w/w base-treated silica was added in 100 g aliquot of the oil. The oil-silica mixtures were incubated in a shaker-incubator at 70° C. for 3 h.

The results in Table 2 show that the treatment decreased the acidity of the water extracts. The pH increased with the amount of added silica product that was used, and silica with 10% Na₂CO₃ (id. D) resulted in larger pH increase compared to silica with 10% KOH (id. B).

TABLE 2

| Formulation + id. | % Base-treated silica in oil | pH of water extract |
|---|---|---|
| 10% KOH, id. B | 1.0 | 5.88 |
| 10% KOH, id. B | 5.0 | 6.37 |
| 10% KOH, id. B | 10.0 | 6.91 |
| 10% KOH, id. B | 25.0 | 7.36 |
| 10% Na₂CO₃, id. D | 1.0 | 6.06 |
| 10% Na₂CO₃, id. D | 5.0 | 7.75 |
| 10% Na₂CO₃, id. D | 10.0 | 8.21 |
| 10% Na₂CO₃, id. D | 25.0 | 8.73 |
| Untreated oil feed | — | 4.06 |

### Example 3

### Neutralization Capacity of Base-Treated Silica Formulations

The purpose of this example was to study the capacity of different base-treated silica formulations to neutralize water soluble acids in a vegetable oil blend.

100 g of the oil blend consisting of 30% palm kernel oil and 70% palm stearine (pH of water extract 4.8) was heated to 70° C. and 0.5% w/w of based-treated silica product as listed in Table 3 was added. The mixture was incubated at 70° C. in a shaker incubator. After 24 h a sample was taken from the oil and the remaining oil decanted without removing any of the silica product. A new 100 g portion of vegetable oil blend was then added and again incubated at 70° for 24 h. This process was repeated for 9 days.

The results in Table 3 show that pH of water extract of the treated oil samples was increased by all formulations. FFA and soap content of the oils are shown in Tables 4 and 5.

The results of this example show that all base-treated silica products efficiently neutralized water-soluble acids in the vegetable oil blend. The results also show that less than 100 ppm soap was formed upon treatment.

TABLE 3
pH of water extract of oil after batch treatment with different base-treated
silica formulations or Lipozyme TLIM immobilized lipase

| | Oil (g)/silica product (g) | | | | | | | |
|---|---|---|---|---|---|---|---|---|
| Formulation | 200 | 400 | 600 | 800 | 1200 | 1400 | 1600 | 1800 |
| 10% KOH, | 7.4 | 6.4 | 6.3 | 6.8 | 6.7 | 6.3 | 6.3 | 6.7 |
| id. B | | | | | | | | |
| 20% KOH, | 7.1 | 6.6 | 6.5 | 7.0 | 6.5 | 6.8 | 6.3 | 7.0 |
| id. E | | | | | | | | |
| 30% KOH, | 7.9 | 7.3 | 7.3 | 7.4 | 7.2 | 7.3 | 7.1 | 6.9 |
| id. G | | | | | | | | |
| 20% Na₂CO₃, | 7.8 | 7.8 | 8.4 | 8.1 | n.a. | 8.3 | 8.3 | 7.5 |
| id. F | | | | | | | | |
| 20% KHCO3, | 8.5 | 7.2 | 6.7 | 7.4 | 6.3 | 7.7 | 6.1 | 7.1 |
| id. H | | | | | | | | |
| 20% K₂CO₃, | 8.9 | 8.1 | 7.7 | 8.3 | 7.2 | 8.6 | 7.4 | 8.0 |
| id. I | | | | | | | | |
| Silica without | 6.6 | 6.6 | 5.8 | 7.0 | 4.8 | 5.9 | 5.4 | 5.6 |
| base | | | | | | | | |
| Lipozyme | 5.5 | 5.9 | 5.1 | 6.1 | 5.3 | 5.2 | 5.0 | 5.1 |
| TL IM | | | | | | | | |

TABLE 4
FFA content of oil after batch treatment with different base-treated
silica formulations or Lipozyme TLIM immobilized lipase

Oil (g)/silica product (g)

| Formulation | 200 | 600 | 1200 | 1800 |
|---|---|---|---|---|
| 10% KOH, id. B | 0.09 | 0.22 | 0.18 | 0.18 |
| 20% KOH, id. E | 0.09 | 0.15 | 0.31 | 0.18 |
| 30% KOH, id. G | 0.07 | 0.13 | 0.16 | 0.16 |
| 20% Na₂CO₃, id. F | 0.07 | 0.15 | 0.16 | 0.18 |
| 20% KHCO3, id. H | 0.14 | 0.14 | 0.21 | 0.16 |
| 20% K₂CO₃, id. I | 0.10 | 0.14 | 0.19 | 0.16 |
| Silica without base | 0.11 | 0.16 | 0.21 | 0.17 |
| Lipozyme TL IM | 0.72 | 0.38 | 0.47 | 0.51 |

TABLE 5
Soap content of oil as ppm sodium or potassium oleate after
batch treatment with different base-treated silica
formulations or Lipozyme TLIM immobilized lipase

Oil (g)/silica product (g)

| Formulation | 200 | 600 | 1200 | 1800 |
|---|---|---|---|---|
| 10% KOH, id. B | 39 | 51 | 22 | 10 |
| 20% KOH, id. E | 105 | 39 | 17 | 29 |
| 30% KOH, id. G | 85 | 25 | 22 | 13 |
| 20% Na₂CO₃, id. F | 72 | 35 | 74 | 17 |
| 20% KHCO₃, id. H | 56 | 73 | 54 | 54 |
| 20% K₂CO₃, id. I | 64 | 78 | 63 | 53 |
| Silica without base | 1 | 2 | 0 | 0 |
| Lipozyme TL IM | 99 | 6 | 0 | 1 |

### Example 4

### Continuous Neutralization of Water Soluble Acids in Oil at Varying Flow Rates

The purpose of this example was to study the contact time required for neutralization of water soluble acids in oil.

The same vegetable oil blend was used as feedstock as in Example 3.

4 g of base-treated silica was packed in two glass columns connected in line. Oil substrate was heated to 70° C. and pumped through the silica product columns with variable flow rate. Oil samples were collected from the untreated feed oil and from the effluent of the 1st and 2nd column.

The results in Table 6 show that all three tested formulations decreased the acidity of the water extract up to a flow rate of at least 20 g oil/g base-treated silica/h. The soap content of the treated oil was in the interval 0 to 53 ppm, corresponding to a maximum of 3.6% of the feedstock FFA (0.13% w/w) being converted to soap.

TABLE 6
pH and soap content at various flow rates for three different
base-treated silica products

| | | | Soap, |
|---|---|---|---|
| | Flow rate g oil/g | pH of water | ppm K-oleate |
| Formulation | silica product/h | extract | or Na-oleate |
| 10% KOH, id. B | 5.3 | 6.4 | 41 |
| 10% KOH, id. B | 6.6 | 6.3 | 31 |
| 10% KOH, id. B | 7.7 | 6.2 | 34 |
| 10% KOH, id. B | 13.5 | 6.2 | 38 |
| 10% KOH, id. B | 19.3 | 6.0 | 41 |
| 20% KOH, id. E | 5.3 | 6.5 | 43 |
| 20% KOH, id. E | 6.1 | 7.0 | 44 |
| 20% KOH, id. E | 7.7 | 5.6 | 47 |
| 20% KOH, id. E | 11.7 | 7.1 | 53 |
| 20% KOH, id. E | 12.5 | 6.8 | 35 |
| 20% KOH, id. E | 14.0 | 6.4 | 44 |
| 20% KOH, id. E | 15.6 | 5.8 | 38 |
| 20% KOH, id. E | 23.6 | 6.4 | 28 |
| 20% KOH, id. E | 29.2 | 5.6 | 27 |
| 20% Na₂CO₃, id. F | 4.6 | 6.3 | 0 |
| 20% Na₂CO₃, id. F | 7.5 | 5.7 | 35 |
| 20% Na₂CO₃, id. F | 12.1 | 5.9 | 45 |
| 20% Na₂CO₃, id. F | 14.8 | 5.9 | 35 |
| 20% Na₂CO₃, id. F | 15.8 | 6.1 | 42 |
| 20% Na₂CO₃, id. F | 26.0 | 5.9 | 36 |
| 20% Na₂CO₃, id. F | 29.9 | 5.7 | 37 |

### Example 5

In this example, water extractable acids in a vegetable oil blend consisting of 30% coconut oil and 70% palm stearine were neutralized using base-treated silica packed in a column and the positive effect on productivity of enzymatic interesterification was investigated. Potassium content in the water extract of the oil was measured to indicate likely soap formation.

20 g of base treated silica (5% KOH content, id. A) was packed into a stainless steel column and vegetable oil pumped through the column at a rate of 3.5 g/g silica product/hour. Samples were collected for the determination of water extract pH and potassium level in the water extract. Once a flow of 2,800 g oil/g silica product had been achieved, the oil exiting the column was collected and pooled for determination of enzyme productivity compared to the oil without silica product pre-treatment. Productivity of the oil was determined in a multiple batch assay in which 0.5 g of Lipozyme TL IM is serially contacted with 100 ml of vegetable oil and the change in SFC measured at 40° C. for each oil batch.

The results in Table 7 show that a silica product containing 5% KOH (id. A) could remove the acidity of at least 3,000 g oil/g material and that the soap level as measured by potassium content in the water extract remained at a low level and did not block the column.

Peroxide value (PV) is a measure of primary oxidation products (hydroperoxides) in oil. These are measured quantitatively on the basis of their ability to liberate iodine from acidic solutions of potassium iodide. This can be measured by titrating with sodium thiosulphate solution or electrochemically. The method used to determine PV is described above.

TABLE 7
Ability of base-containing silica to neutralise oil

| | Peroxide | pH of | Potassium level |
|---|---|---|---|
| Flow | Value | water | in water extract |
| g oil/g silica product | Meq/kg | extract | ppm |
| 0 | 0.68 | 4.2 | 0.68 |
| 200 | 0.71 | 6.8 | 0.06 |
| 487 | 0.78 | 6.9 | 0.41 |
| 645 | 0.7 | 7.3 | 0.92 |
| 729 | 0.41 | 7.1 | 0.62 |
| 1087 | 0.94 | 6.7 | 0.42 |
| 1112 | 0.89 | 6.8 | 0.37 |
| 1165 | 0.78 | 6.8 | 0.33 |
| 1192 | 0.88 | 6.9 | 0.34 |
| 1304 | 0.81 | 6.7 | 0.30 |
| 1375 | 0.88 | 6.6 | 0.29 |
| 1463 | 0.7 | 6.5 | 0.35 |
| 1495 | 1.03 | 6.8 | 0.30 |
| 1559 | 0.88 | 6.8 | 0.34 |
| 1591 | 0.52 | 6.8 | 0.19 |
| 1657 | 0.61 | 6.6 | 0.25 |
| 1719 | 1.23 | 6.7 | 0.48 |
| 1786 | 1.04 | 6.7 | 0.24 |
| 1890 | 0.84 | 6.8 | 0.21 |
| 1922 | 0.89 | 6.8 | 0.26 |
| 1992 | 0.68 | 6.7 | 0.17 |
| 2023 | 0.69 | 6.9 | 0.25 |
| 2093 | 0.7 | 6.8 | 0.15 |
| 2252 | 0.88 | 6.4 | 0.30 |
| 2360 | 0.87 | 6.8 | 0.32 |
| 2536 | 0.78 | 6.9 | 0.40 |
| 2634 | 1.2 | 6.7 | 0.30 |
| 2989 | 0.97 | 6.6 | |

Productivity of Lipozyme TL IM was determined using oil collected from the column and then pooled. This was compared to untreated oil using the MBA assay.

TABLE 8
Productivity of treated and untreated oils

| | | pH of | | |
|---|---|---|---|---|
| | Peroxide | water | Avg. prod. | V 1/ |
| Oil Blend, | Value | extract | [kg/kg/hr] | [kg/kg] |
| Feed (Oil without treatment) | 0.6-1.2 | 4.2 | 1.38 | 909 |
| Oil after treatment | 0.6-1.2 | 6.4-6.7 | 10.43 | 2214 |

The results in Table 8 show that after 2,800 g oil/g silica product have passed through the column the resulting oil is able to support a drastically higher productivity than the untreated oil. The V½ measure (the amount of oil passing over the enzyme to reduce initial activity by 50%) increases considerably as does the calculated average flow to give 90% of full conversion.

### Example 6

In this example, water extractable acids in a vegetable oil blend were neutralized using base-treated silica packed in a column and the positive effect on productivity of enzymatic interesterification was investigated.

Two small scale (aquarium) continuous enzyme reactors were used to investigate the acid neutralizing affect of the base treated silica containing 10% KOH (id. B). In the first reactor 10 g of base treated silica was filled into one column and 20 g of Lipozyme TL IM, filled into a second column connected in series to the first one. In the second reactor a single column was filled with 20 g of Lipozyme TL IM. A vegetable oil blend (70% palm stearine+30% coconut oil) was pumped through the columns at a flow rate of 1.5-2.5 g oil/g enzyme/hour and at a temperature of 70° C.

Samples were collected on a daily basis for the determination of water extract pH and SFC (40° C.) to determine if the oil was neutralized and enzyme performance increased. The pressure drop across the columns was monitored to determine if soap formation was leading to column blockage. The oil blend used had a water extract pH of 4.9.

The results (Table 9) show that as oil passes down the column containing only enzyme, activity is lost and the SFC value starts to return to that of the unconverted oil SFC (=13.8 at 40° C.). When the base treated silica material is used as a pre-column the residual acidity is removed and the enzyme activity is maintained for a longer period. At a throughput of ˜750 g oil/g enzyme without the pre-column, enzyme activity is reduced and the SFC value increases. With the pre-column, enzyme activity is maintained until at least 2,100 g oil/g enzyme. The pressure drop across both enzyme columns was monitored and during the course of the trial did not exceed 0.5 bar indicating no build up of soap which would have resulted in a pressure increase.

TABLE 9
Comparison of performance of enzyme alone and enzyme plus pre-column

| | |
|---|---|
| Enzyme | Enzyme + pre- |
| Alone | column |

| g oil/g | % SFC | g oil/g | % SFC |
|---|---|---|---|
| enzyme | (40° C.) | enzyme | (40° C.) |
| 70.7 | 6.3 | 62.1 | 6.2 |
| 190.4 | 6.4 | 153.7 | 6.7 |
| 298.9 | 6.2 | 241.7 | 6.6 |
| 332.3 | 5.9 | 275.2 | 6.5 |
| 366.6 | 6.3 | 315.2 | 6.7 |
| 413.6 | 6.1 | 365.0 | 6.5 |
| 463.9 | 6.6 | 415.8 | 5.8 |
| 513.2 | 6.3 | 466.7 | 5.7 |
| 561.7 | 6.7 | 533.5 | 6.1 |
| 623.4 | 7.0 | 587.2 | 6.5 |
| 668.6 | 7.6 | 642.4 | 6.8 |
| 712.5 | 8.0 | 715.6 | 8.1 |
| 770.5 | 9.4 | 823.9 | 8.1 |
| 862.7 | 10.1 | 863.1 | 8.2 |
| 896.4 | 10.4 | 914.7 | 8.0 |
| 940.0 | 10.4 | 945.4 | 7.8 |
| 982.6 | 10.5 | 992.7 | 8.2 |
| 1012.6 | 10.6 | 1048.2 | 8.0 |
| 1063.8 | 10.4 | 1176.4 | 8.2 |
| 1169.6 | 10.8 | 1209.4 | 8.1 |
| 1197.8 | 11.1 | 1282.0 | 7.0 |
| 1255.2 | 9.9 | 1319.4 | 7.0 |
| 1296.4 | 10.3 | 1375.0 | 7.3 |
| 1357.6 | 11.1 | 1450.7 | 7.5 |
| 1437.3 | 11.8 | 1491.5 | 7.3 |
| 1482.0 | 11.3 | 1549.4 | 7.6 |
| 1542.5 | 11.3 | 1608.8 | 7.7 |
| 1603.1 | 11.9 | 1687.7 | 7.5 |
| 1683.3 | 11.6 | 1849.7 | 7.9 |
| 1845.3 | 12.3 | 1906.9 | 8.2 |
| 1902.6 | 12.1 | 1966.4 | 8.1 |
| 1961.8 | 12.1 | 2025.7 | 8.4 |
| 2021.1 | 12.3 | 2107.0 | 8.5 |

### Example 7

In this example, the effect on enzyme productivity of a pre-column of 10% KOH base treated silica is compared to direct admixture of the material with Lipozyme TL IM.

Three aquarium reactors were set up to operate in parallel using an oil blend containing 70% Palm stearine and 30% Palm kernel oil. The oil blend had a water extract pH of 4.6.

In the first aquarium, two columns were filled with Lipozyme TL IM (˜5 g in each column). In the second aquarium, a pre-column containing ˜5 g of 10% KOH (id. B) treated silica was operated in series with a second column containing ˜5 g of lipozyme TL IM. For the third aquarium, a mixture of 20% by weight base treated silica and 80% Lipozyme TL IM was prepared and filled into 2 columns, each containing ˜6.25 g of material. These were connected in series. A vegetable oil blend was pumped at 70° C. through the columns in the three aquaria at a flow rate equivalent to 6 g oil/g enzyme/hour based on the enzyme amount in the first aquarium reactor.

Samples were collected on a daily basis for the determination of SFC (40° C.) and the pressure drop across the columns was monitored to determine if soap formation was leading to column blockage.

In Table 10 below, SFC is shown for oil which has passed through both columns of the first (enzyme alone) and third (admixture enzyme+silica) aquarium.

The results for the aquarium reactor containing only enzyme show that activity is lost as a result of the inorganic acid contained within the oil as the SFC returns quickly towards the start position (16.4% at 40° C.).

When the enzyme and base treated silica are mixed together, the SFC values take longer to return to the start value, indicating that the enzyme activity is being maintained over a longer processing period.

TABLE 10
Comparison of treatment options - enzyme alone or
admixture enzyme + silica

| | |
|---|---|
| | Admixture enzyme + |
| Enzyme alone | Silica |

| g oil/g | | g oil/g | |
|---|---|---|---|
| enzyme | SFC 40° C. | ezyme | SFC 40° C. |
| 194 | 9.8 | 195 | 8.2 |
| 405 | 12.5 | 381 | 9.4 |
| 554 | 12.7 | 576 | 10.8 |
| 974 | 13.2 | 1015 | 11.7 |
| 1098 | 13.4 | 1147 | 11.6 |
| 1215 | 13.9 | 1266 | 11.6 |
| 1333 | 14.2 | 1392 | 11.9 |
| 1466 | 14.2 | 1522 | 12.4 |
| 1849 | 14.8 | 1927 | 13.4 |
| 1965 | 15.1 | 2043 | 13.6 |
| 2085 | 14.5 | 2165 | 12.9 |
| 2209 | 14.4 | 2286 | 13.4 |
| 2333 | 14.7 | 2407 | 13.7 |
| 2459 | 15.0 | 2532 | 14.6 |

In Table 11 below, SFC for oil which has passed through one column of the first aquarium (enzyme alone) is compared to SFC for oil which has passed through the pre-column and the enzyme column of the second aquarium.

When enzyme alone is compared to the situation where a pre-column of base treated silica is used before the enzyme column, the SFC value returns quickly towards the start position in the enzyme only column. Whereas in the situation where the oil first passes through a pre-column of base treated silica, it is clear that the enzyme activity is maintained for considerably longer.

Comparing admixture of base treated silica and enzyme with the situation with a separate precolumn followed by enzyme, it is clear that for the same approximate volume of oil passing (˜2,500 g oil/g enzyme), the pre-column provides the best retention of enzyme activity.

After passage of ˜4,300 g oil/g enzyme the pressure drop remained below 1 bar indicating no significant build up of soap within the column which would have lead to blockage of the column.

TABLE 11
Comparison of treatment options - enzyme alone or
silica pre-column + enzyme

| | |
|---|---|
| | Pre-column silica + |
| Enzyme alone | enzyme |

| g oil/g | | g oil/g | |
|---|---|---|---|
| enzyme | SFC 40° C. | enzyme | SFC 40° C. |
| 405 | 14.0 | 418 | 11.3 |
| 842 | 14.9 | 852 | 12.1 |
| 1198 | 15.2 | 1214 | 12.6 |
| 2089 | 15.6 | 2122 | 12.7 |
| 2382 | 15.4 | 2354 | 12.4 |
| 2653 | 15.6 | 2638 | 12.5 |
| 2924 | 15.6 | 2914 | 13.1 |
| 3227 | 15.5 | 3236 | 13.2 |
| 4036 | 15.8 | 4071 | 13.8 |
| 4302 | 15.9 | 4338 | 13.6 |
| 4384 | 15.3 | 4414 | 13.4 |
| 4654 | 15.2 | 4677 | 13.4 |
| 4935 | 15.4 | 4951 | 13.8 |
| 5210 | 15.9 | 5228 | 14.0 |

### Example 8

In this example, the effect on enzyme productivity of a precolumn of 20% Na₂CO₃ base treated silica was investigated.

Two aquarium reactors were set up to operate in parallel using an oil blend containing 70% palm stearine and 30% palm kernel oil. The oil blend had water extract pH of 4.7. In the first aquarium, two columns were filled with Lipozyme TLIM (6 g in each column). In the second aquarium, a pre-column containing 3 g of 20% Na₂CO₃ (id. F) treated silica was operated in series with two columns containing Lipozyme TLIM (6 g in each column).

The vegetable oil blend was pumped at 70° C. through the columns in the two aquaria at a flow rate equivalent to 3 g oil/g enzyme/hour based on the total enzyme amount in the columns. Samples were collected daily from the outlet of the precolumn in aquarium 2 for the determination of water extract pH and from the outlet of the last Lipozyme TLIM column of both aquaria for the determination of SFC (40° C.). These data are shown in table 12 below.

TABLE 12
Comparison of performance of enzyme alone and enzyme plus pre-column

| | | Enzyme + precolumn with silica i.d. F | | |
|---|---|---|---|---|
| Enzyme alone | | (20% Na₂CO₃) | | |
| g oil/g | | | Water extract pH of oil | SFC |
| enzyme | SFC 40° C. | g oil/g enzyme | after precolumn | 40° C. |
| 95 | 4.8 | 90 | 5.6 | 4.6 |
| 156 | 4.6 | 157 | 5.5 | 4.3 |
| 206 | 4.5 | 233 | 6.2 | 4.9 |
| 383 | 6.3 | 455 | 6.5 | 6.5 |
| 435 | 7.1 | 520 | 6.6 | 7.0 |
| 490 | 8.0 | 589 | nd | 7.3 |
| 549 | 8.9 | 663 | nd | 7.7 |
| 606 | 9.2 | 735 | 6.8 | 8.0 |
| 744 | 9.9 | 754 | 6.5 | 8.9 |
| 793 | 10.9 | 966 | 7.7 | 9.5 |
| 853 | 11.8 | 1036 | 6.0 | 9.8 |
| 909 | 11.7 | 1094 | 5.8 | 9.9 |
| 961 | 12.5 | 1146 | 6.2 | 10.3 |
| 1139 | 15.6 | 1327 | 6.1 | 11.6 |
| — | — | 1384 | 6.0 | 11.6 |

The results show that as oil passes through the precolumn with base-treated silica material the water extract pH of oil is increased from the initial value 4.7.

The SFC value of the processed oil increases as more oil is passed through the enzyme columns, indicating loss of enzyme activity. The loss of activity occurs at a relatively lower amount of oil per g enzyme, showing that the neutralization of oil in the precolumn improved the lifetime of the enzyme.

1-15. (canceled)

16. A method for treatment of oil containing water-soluble acids comprising the steps of:
a) contacting oil with particles of base-containing porous amorphous silica having an average particle size of above 150 micrometer, and
b) reacting said oil with a lipolytic enzyme.

17. The method of claim 16, wherein steps a) and b) are carried out sequentially or simultaneously.

18. The method of claim 16, wherein step b) is performed as a process where the oil is passed through a treatment system comprising at least one reactor containing lipolytic enzyme, and wherein the particles of base-containing porous amorphous silica are loaded in at least one reactor connected in series to the at least one reactor containing lipolytic enzyme.

19. The method of any claim 16, wherein step b) is performed as a process where the oil is passed through a treatment system comprising at least one reactor containing lipolytic enzyme, and wherein the particles of base-containing porous amorphous silica are mixed with the lipolytic enzyme in at least one such reactor.

20. The method of claim 16, wherein step b) is performed as a process where the oil is passed through a treatment system comprising at least one reactor containing lipolytic enzyme, and wherein the particles of base-containing porous amorphous silica are loaded on top of the lipolytic enzyme in at least one such reactor.

21. The method of claim 16, wherein step a) and b) are carried out in batch process or in continuous mode of operation or any combination of these.

22. The method of claim 16, wherein the reaction with the lipolytic enzyme results in interesterification or transesterification of lipids in the oil.

23. The method of claim 16, wherein the lipolytic enzyme is immobilized.

24. The method of claim 16, wherein the amorphous silica is precipitated silica.

25. The method of claim 16, wherein the particles of base-containing porous amorphous silica have a moisture content of less than 30%.

26. The method of claim 16, wherein the particles of base-containing porous amorphous silica have a surface area of 10-1,200 m²/g.

27. The method of claim 16, wherein the amount of base in the particles of base-containing porous amorphous silica is 0.5-50 wt. %.

28. The method of claim 16, wherein the base is selected from the group consisting of sodium carbonate (Na₂CO₃), sodium hydrogen carbonate (NaHCO₃), potassium carbonate (K₂CO₃), potassium hydrogen carbonate (KHCO₃), sodium hydroxide (NaOH), potassium hydroxide (KOH), and any combination thereof.

29. The method of claim 16, wherein the oil is a vegetable oil.

30. Particles of base-containing porous amorphous silica having the following properties:
a) an average particle size of above 150 micrometer,
b) an average pore diameter of 20-5,000 Angstroms,
c) a surface area of 10-1,200 m²/g, and
d) a moisture content of less than 30%,
wherein the amount of base is 0.5-50 wt. %.
